(19) United States
(12) Patent Application Publication
(10) Pub. No.: US 2023/0000344 A1
(43) Pub. Date: Jan. 5, 2023

(54) OPHTHALMOLOGY INSPECTION DEVICE AND PUPIL TRACKING METHOD

(71) Applicant: Medimaging Integrated Solution, Inc., Hsinchu (TW)

(72) Inventors: Yu Chian Lin, Hsinchu (TW); Jyun-Hong Li, Hsinchu (TW); Yung-En Kuo, Hsinchu (TW); Yu-Tsung Lee, Hsinchu (TW)

(21) Appl. No.: 17/854,607

(22) Filed: Jun. 30, 2022

(30) Foreign Application Priority Data
Jun. 30, 2021 (TW) 110124054

(51) Int. Cl. A61B 3/113; G06T 7/136; G06T 7/13; G06T 3/40; G06T 5/00; G06T 5/20; G06T 7/60; G06T 7/246; H04N 5/225; H04N 5/232; A61B 3/14; A61B 3/00

(52) U.S. Cl. CPC A61B 3/113; G06T 7/136; G06T 7/13; G06T 3/40; G06T 5/002; G06T 5/20; G06T 7/60; G06T 7/246; H04N 5/2256; H04N 5/23212; A61B 3/14; A61B 3/0008; G06T 2207/20028; G06T 2207/20032; G06T 2207/20036; G06T 2207/30041

(57) ABSTRACT

A pupil tracking method includes: retrieving an external eye image of a subject, wherein the external eye image includes a pupil of the subject; performing an image preprocessing on the external eye image, wherein the image preprocessing includes performing a binary conversion on the external eye image to obtain a binary image; finding out a contour boundary of each feature in the binary image, and finding out a pupil feature based on a variance of a distance from the contour boundary of each feature to a corresponding reference point; fitting the contour boundary of the pupil feature by a boundary fitting method to find a center coordinate of the pupil feature. The abovementioned pupil tracking method can track the pupil of the subject's eyeball without using a stereo camera. An ophthalmology inspection device using the abovementioned pupil tracking method is also disclosed.

### BACKGROUND OF THE INVENTION

### 1. Field of the Invention

The present invention relates to an inspection device, particularly to an ophthalmology inspection device and a pupil tracking method.

### 2. Description of the Prior Art

An ophthalmology inspection device is used to obtain the physiological information of the eyes of a subject. The physician will diagnose the disease based on the physiological information of the eyes. For example, a fundus camera can capture the images of a fundus; a tonometer can measure intraocular pressure; a corneal topography device can measure the topography of the surface of an eyeball; a refractometer can measure the diopter of an eyeball. While inspecting, the abovementioned ophthalmology inspection devices need to be aligned to the eyeball of the subject to acquire better inspection results. For example, a fundus camera needs to project the illumination light to the fundus through the pupil of the eyeball of a subject; the light reflected from the fundus enters the imaging system through the pupil to form images of the fundus. Therefore, the imaging system of a fundus camera needs to be exactly aligned to the pupil so as to acquire a larger view field and a better fundus image.

A conventional fundus camera uses a stereo camera to recognize the pupil and acquire the coordinates of the center of the pupil. Then, a motor drives the optical lens to make the optical axis of the imaging system be coaxial with the pupil. Because of the stereo camera, the conventional fundus camera is more expensive, more bulky and heavier. Thus, the conventional fundus camera can only be used as a desk-top apparatus.

Accordingly, it has become a target the concerned fields are eager to achieve: using a simple and compact design to enable ophthalmology inspection devices to track the pupil of a subject.

### SUMMARY OF THE INVENTION

The present invention provides an ophthalmology inspection device and a pupil tracking method, wherein a pupil feature is found out from the external eye image, and a boundary fitting method is used to find a center coordinate of the pupil feature, whereby the present invention can track the pupil of the eyeball of a subject without using a stereo camera.

In one embodiment, the pupil tracking method of the present invention comprises steps: using an ophthalmology inspection device to acquire an external eye image of a subject, wherein the external eye image includes a pupil of the subject; using the ophthalmology inspection device to perform an image preprocessing on the external eye image, wherein the image preprocessing includes performing a binary conversion on the external eye image to obtain a binary image; finding out a contour boundary of each feature in the binary image, and finding out a pupil feature based on a variance of a distance from the contour boundary of each feature to a corresponding reference point; fitting the contour boundary of the pupil feature in a boundary fitting method to find a center coordinate of the pupil feature.

In one embodiment, the ophthalmology inspection device of the present invention comprises an illumination element, an image sensor, an imaging lens assembly and a signal processing element. The illumination element generates an illumination light beam to illuminate an external eye region of a subject. The image sensor receives the light beam reflected from the external eye region to generate an external eye image, wherein the external eye image includes a pupil of the subject. The imaging lens assembly is disposed at a light-input side of the image sensor to condense the reflected light and form an image to the image sensor. The signal processing element is electrically connected with the image sensor and performs a pupil tracking method. The pupil tracking method comprises steps: acquiring the external eye image output by the image sensor; performing an image preprocessing on the external eye image, wherein the image preprocessing includes performing a binary conversion on the external eye image to obtain a binary image; finding out a contour boundary of each feature in the binary image, and finding out a pupil feature based on a variance of a distance from the contour boundary of each feature to a corresponding reference point; fitting the contour boundary of the pupil feature in a boundary fitting method to find a center coordinate of the pupil feature, and calculating the deviation between an optical axis of the imaging lens assembly and the pupil of the subject according to the center coordinate of the pupil feature.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein

FIG. 1 is a diagram schematically showing a fundus camera according to one embodiment of the present invention;

FIG. 2 is a flowchart of a pupil tracking method according to one embodiment of the present invention;

FIG. 3 is a flowchart of an image preprocessing of a pupil tracking method according to one embodiment of the present invention;

FIGS. 4a-4d are images relating to a pupil tracking method according to one embodiment of the present invention;

FIGS. 5a and 5b are indication signals of a pupil tracking method according to one embodiment of the present invention;

FIG. 6 is a diagram schematically showing a tonometer according to one embodiment of the present invention;

FIG. 7 is a diagram schematically showing a corneal topography device according to one embodiment of the present invention; and

FIG. 8 is a diagram schematically showing an automatic refractometer according to one embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The ophthalmology inspection device of the present invention uses an imaging system to acquire an external eye image of a subject and uses a pupil tracking method to find out a pupil feature from the external eye image and the center coordinate of the pupil, whereby the ophthalmology inspection device may be exactly aligned to the pupil of the subject. Below, a fundus camera is used to demonstrate the ophthalmology inspection device and the pupil tracking method of the present invention.

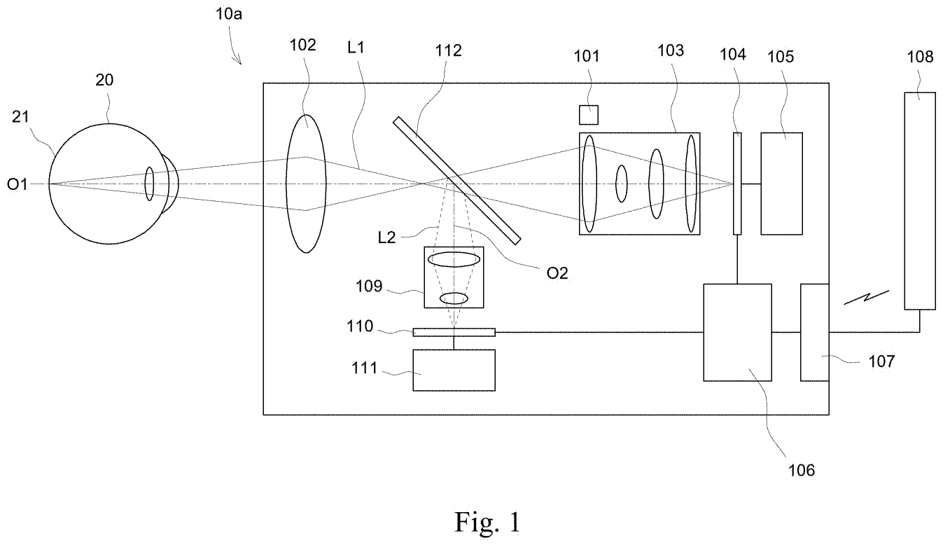

Refer to FIG. 1. In one embodiment of the present invention, a fundus camera 10a comprises an illumination element 101, an imaging lens assembly 103, an image sensor 104 and a signal processing element 106. The illumination element 101 generates an illumination light beam to illuminate an external eye region of an eyeball 20 of a subject. In the embodiment of the fundus camera 10a, the illumination element 101 generates an illumination light beam. The illumination light beam is condensed by an object lens 102, then passing through the eyeball 20 and illuminating a fundus 21. In the embodiment shown in FIG. 1, the illumination element 101 is deviated from an optical axis O1 of the imaging lens assembly 103. However, the present invention is not limited by FIG. 1. In other embodiments, the illumination element may provide annular illumination light. In one embodiment, the illumination element includes a condenser lens, a baffle board with an annular opening, a relay lens, and a reflector with a circular opening, whereby to provide annular illumination light. In one embodiment, the illumination element 101 includes at least one visible light-emitting element and at least one infrared light-emitting element. The infrared light generated by the infrared light-emitting element may be used to search the fundus. The visible light generated by the visible light-emitting element nay be used as the light source for photography. Besides, the visible light or the infrared light may be used as the illumination light for photographing the external eye region.

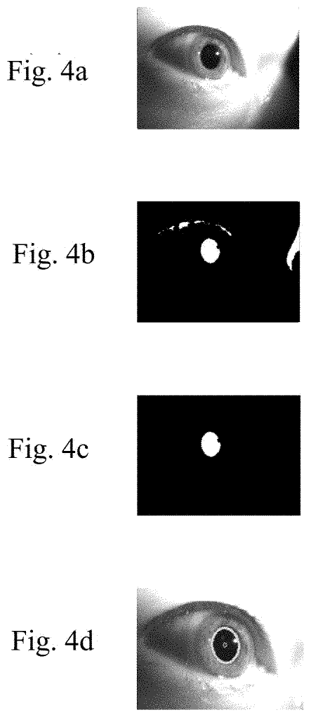

The imaging lens assembly 103 is disposed at the light-input side of the image sensor 104, condensing the light reflected by the external eye region and forming an image to the image sensor 104. The image sensor 104 receives the light reflected by the external eye region to generate an external eye image, wherein the external eye image includes a pupil of a subject, as shown in FIG. 4a. In one embodiment, the fundus camera 10a of the present invention further comprises an imaging focal length-adjusting element 105. In cooperation with the imaging focal length-adjusting element 105, the fundus camera may be operated to make the reflected light L1 of the external eye region or the fundus of the eyeball 20 image on the image sensor. For example, the imaging focal length-adjusting element 105 may use an electric motor or a mechanical structure to drive the image sensor 104 to move physically along the optical axis O1, whereby to adjust the focal length. Alternatively, the imaging focal length-adjusting element 105 may use an electric motor or a mechanical structure to drive at least one lens of the imaging lens assembly 103 to move physically along the optical axis O1, whereby to adjust the focal length. In one embodiment, the imaging lens assembly 103 includes at least one liquid-state lens; the imaging focal length-adjusting element 105 may adjust the curvature of the liquid-state lens of the imaging lens assembly 103 to adjust the focal length.

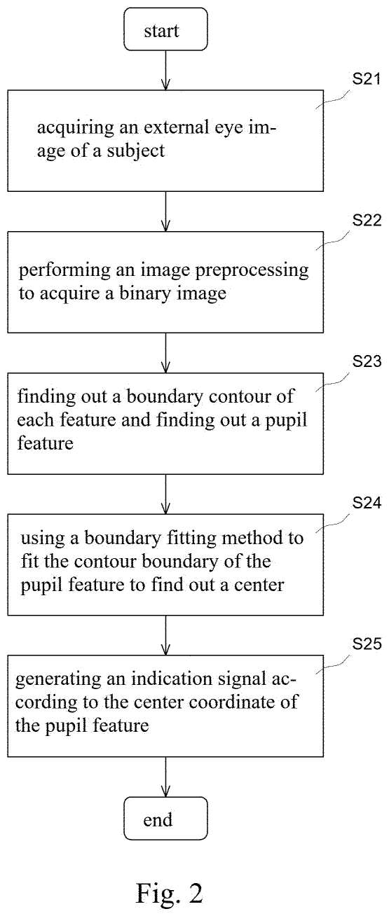

The signal processing element 106 is electrically connected with the image sensor 104 and executes a pupil tracking method according to the external eye image output by the image sensor 104 to find out the pupil and the center coordinate of the pupil. Refer to FIG. 2 for a flowchart of a pupil tracking method according to one embodiment of the present invention. In Step S21, the signal processing element 106 acquires an external eye image output by the image sensor 104. In one embodiment, the features, such as the pupil and the sclera, would not be underexposed or overexposed under an appropriate illumination condition or an appropriate exposure condition, as shown in FIG. 4a.

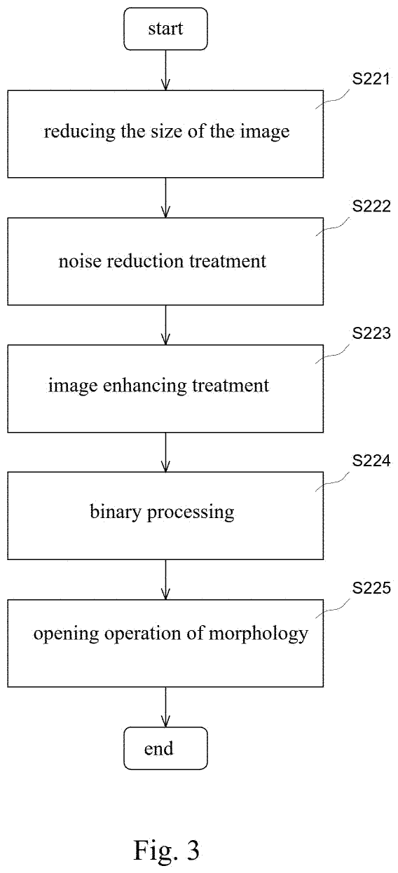

Next, in Step S22, perform an image preprocessing, such as a binary conversion, to acquire a binary image. In one embodiment, the image preprocessing step (Step S22) further comprises a plurality of image processing steps, whereby to reduce the computation amount of the image processing or raise the quality of the binary image. Refer to FIG. 3 for a flowchart of an image preprocessing according to one embodiment of the present invention. Firstly, in Step S221, compress the size of the external eye image to reduce the computation amount of the succeeding image processing. For example, each of the length and width of the external eye image is reduced to a half of the original size, whereby the computation amount may be reduced to a quarter of the original computation amount. Next, in Step S222, perform a noise reduction treatment of the external eye image, whereby to eliminate the grain-like noises of the external eye image. The grain-like noises mainly originate from the image sensor. For example, while the image sensor is affected by the ambient temperature, the image is likely to have grain-like noises. In one embodiment, the noise reduction device may be an average filter, a Gaussian filter, a median filter, or a bilateral filter. The algorithms of all the abovementioned noise reduction devices perform a smooth operation of the signals of the image. Among them, the bilateral filter and the median filter can preserve the boundaries of the image. Thus, the bilateral filter and the median filter are suitable to be used in the images whose boundary information is important. For example, the median filter uses the center of a window having a size of m×n to scan all the pixels of the external eye image; for each pixel, the median of the gray levels of the corresponding m×n window is output to function as a new gray level of the pixel, whereby the gray levels of neighboring pixels are similar, wherefore is achieved the effect of eliminating noises.

Next, in Step S223, perform an image enhancing treatment of the external eye image to enhance the boundary of the image. The image enhancing treatment may enhance the difference of the gray levels of the image boundary. In other words, the image enhancing treatment may enhance the boundary of the pupil feature. Thereby, while the pupil feature is extracted, the complete information of the boundary of the pupil can be acquired. Further, the image enhancing treatment may adapt to the variance resulting from different photographing environments or different races. In one embodiment, the image enhancing treatment may be realized by a gamma correction technology, a histogram equalization technology, a homomorphic filter technology, an unsharp masking technology, or a combination thereof. For example, the unsharp masking technology performs a Gaussian filtering treatment or a low-pass filtering treatment on the external eye image to acquire a blurred image, subtracts the blurred image from the original image to acquire a boundary image, and performs a linear combination of the boundary image and the original image to acquire a boundary-enhanced image.

Next, in Step S224, perform a binary conversion on the external eye image to acquire a binary image. According to the extent of boundary enhancement by the unsharp masking technology, select an optimized value as the threshold t of the binary conversion. If the gray level p of a pixel after boundary enhancement is greater than the threshold t, assign a value of 255 to the gray level of the pixel. If the gray level p of a pixel after boundary enhancement is smaller than the threshold t, assign a value of 0 to the gray level of the pixel. After the binary conversion of all the pixels is completed, a binary image is obtained.

It is easily understood: after the binary conversion, defects (such as boundary discontinuities) or small-area noises may appear. In one embodiment, an opening operation of morphology (Step S225) is performed on the binary image generated by Step S224 to compensate for defects or small-area noises, which are generated in the binary conversion. The opening operation of morphology firstly performs erosion on the binary image to remove small-area noises and then performs dilation on the binary image to restore the shape, whereby to acquire a noise-filtered and revamped binary image, as shown in FIG. 4b.

Return to the description of the pupil tracking method shown in FIG. 2. After the binary image has been acquired, the process proceeds to finding out the boundary contour of each feature and finding out a pupil feature in Step S23. It is easily understood: the binary image includes non-pupil features, such as eyelashes, an eyelid, and shadows, as shown in FIG. 4b. Therefore, it is necessary to find out a pupil feature from the contour boundaries of the features of the binary image. In one embodiment, the pupil feature is found out according to the variance of the distance between the boundary contour of each feature and a corresponding reference point. In one embodiment, a contour searching method is used to acquire the contour boundary of each feature from the binary image, i.e. the information of the coordinates of the boundary contour, and stores the information in a vector-type point set of a 2D coordinate system. Next, a minimum enclosing circle method is used to perform circle fitting on the contour shape of each feature. For example, an iteration method is used to find a minimum circle able to enclose a given 2D-coordinate point set, whereby each feature may acquire a minimum enclosing circle covering the contour of the feature, and whereby the center and radius of the minimum enclosing circle are also acquired. Then, use the center of the minimum enclosing circle as a reference point, and calculate the variance of the distance between the reference point and the contour boundary of the corresponding feature. It is easily understood: the more the contour boundary of a feature approaches a circle, the more the variance of the distance between the reference point and the contour boundary of the feature approaches zero. Therefore, an appropriate preset value may be used to filter out non-pupil features and find out a pupil feature, as shown in FIG. 4c. For example, while the variance of the distance between the reference point and the contour boundary of a feature is smaller than or equal to 3, the feature is a pupil feature.

Next, in Step S24, use a boundary fitting method to fit the contour boundary of the pupil feature found in Step S23 to find out a center coordinate of the pupil feature, as shown in FIG. 4d. In one embodiment, the boundary fitting method may be a circle fitting method, an ellipse fitting method, or a minimum enclosing circle method. After the fitting shape of the contour boundary of the pupil feature is acquired, the center of the fitting shape can be worked out to obtain the center coordinate of the pupil feature. In one embodiment, a least square method is used to calculate the center of the fitting shape. In the example using the ellipse fitting method, calculating the least sum of the square distances between the center of the ellipse and the coordinate points of the boundary can obtain a set of center and radius, which have the minimum error, whereby the pupil can be precisely tracked. According to the center coordinate of the pupil feature, the signal processing element 106 may work out the deviation between an optical axis of the imaging lens assembly and the eyeball 20 of a subject. According to the deviation, a motor (not shown in the drawing) automatically drives the imaging system to be exactly aligned to the pupil of the eyeball 20.

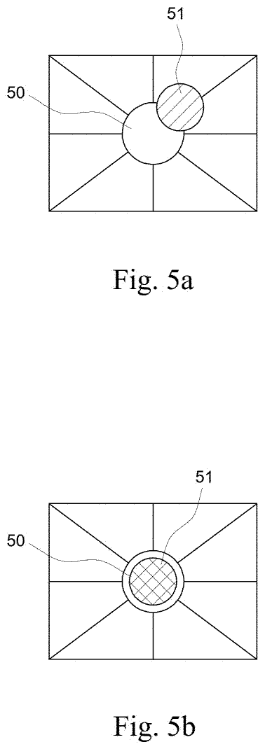

Refer to FIG. 2. In one embodiment, the signal processing element 106 generates an indication signal according to the deviation (Step S25). According to the indication signal, the operator may adjust the relative position of the ophthalmology inspection device and the subject to make the ophthalmology inspection device be exactly aligned to the pupil of the eyeball of the subject. Refer to FIG. 1, FIG. 5a and FIG. 5b. In one embodiment, the fundus camera 10a further comprises an external display device 108. The external display device 108 is coupled to the signal processing element 106 through a communication interface 107 in a wired or wireless way. According to the calculated deviation, the signal processing element 106 presents an indication signal 51 on the external display device 108. The indication signal 51 will guide the operator to adjust the relative position of the ophthalmology inspection device and the subject. As shown in FIG. 5b, while the indication signal 51 coincides with a preset position 50, it means that the ophthalmology inspection device is exactly aligned to the pupil of the eyeball 20 of the subject. In one embodiment, while the indication signal 51 coincides with the preset position 50, the indication signal 51 changes its color to remind the operator to capture the image of the fundus. Through the abovementioned structure, an inexperienced operator can also operate the fundus camera 10a of the present invention to capture the image of the fundus of the subject. Even though the fundus camera 10a is in a handheld form, it can also acquire superior fundus images.

In one embodiment, the subject himself may operate the ophthalmology inspection device of the present invention according to the indication signal 51. Refer to FIG. 1 once again. In one embodiment, the fundus camera 10a of the present invention further comprises a display lens assembly 109, an internal display device 110, a display focal length-adjusting element 111, and a light splitter 112. The internal display device 110 is electrically connected with the signal processing element 106. According to the calculated deviation, the signal processing element 106 presents the indication signal 51 on the internal display device 110. The display lens assembly 109 is disposed at the light-output side of the internal display device 110. The display focal length-adjusting element 111 is connected with the internal display device 110 or the display lens assembly 109. The display focal length-adjusting element 111 may drive the internal display device 110 to move along an optical axis O2 of the display lens assembly 109 or adjust the position or curvature of the lens of the display lens assembly 109, whereby to adjust the focal length of the indication signal 51 to make the light beam L2, which is generated by the internal display device 110, form an image on the fundus 21 of the tested eyeball 20. The light splitter 112 is optically coupled to the internal display device 110 and the imaging lens assembly 103, making the indication signal 51 be imaged on the fundus 21 of the tested eyeball 20. According to the indication signal 51 presented by the internal display device 110, the subject may adjust the relative position of the subject and the fundus camera 10a by himself. For example, the subject may move his own position or the position of the fundus camera 10a to make the fundus camera 10a be exactly aligned to the pupil of the subject. Through the abovementioned structure, a subject himself can also operate the fundus camera 10a of the present invention. Even though the fundus camera 10a is in a handheld form, it can also acquire superior fundus images.

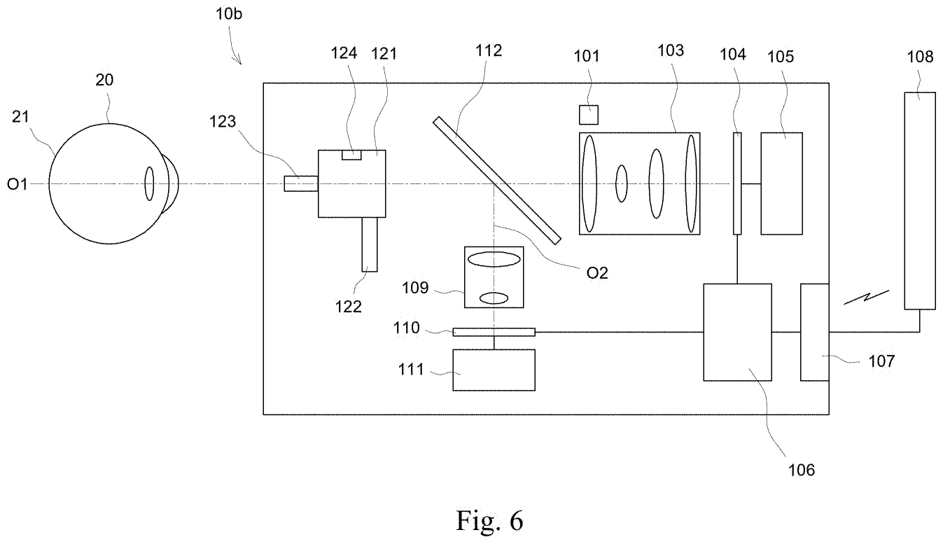

The pupil tracking method of the present invention is applicable to different ophthalmology inspection devices. Refer to FIG. 6 for a fundamental structure of a tonometer 10b. The main functional elements of the tonometer 10b include a chamber 121, a pressure source 122, an air injector 123 and a pressure sensor 124. The pressure source 122 supplies air to the chamber 121 to make the chamber have an appropriate pressure. The air injector 123 spurts air to the tested eyeball 20. The pressure sensor 124 measures the pressure inside the chamber 121, whereby to calculate the intraocular pressure of the tested eyeball 20. The other elements of the tonometer 10b shown in FIG. 6, which have the same numeral symbols as the elements in FIG. 1, also have the same functions as these elements in FIG. 1. Since the technical contents thereof has been described before, it will not repeat herein. The persons having ordinary knowledge of the art should be able to modify the pupil tracking method and the ophthalmology inspection devices without departing from the spirit of the present invention.

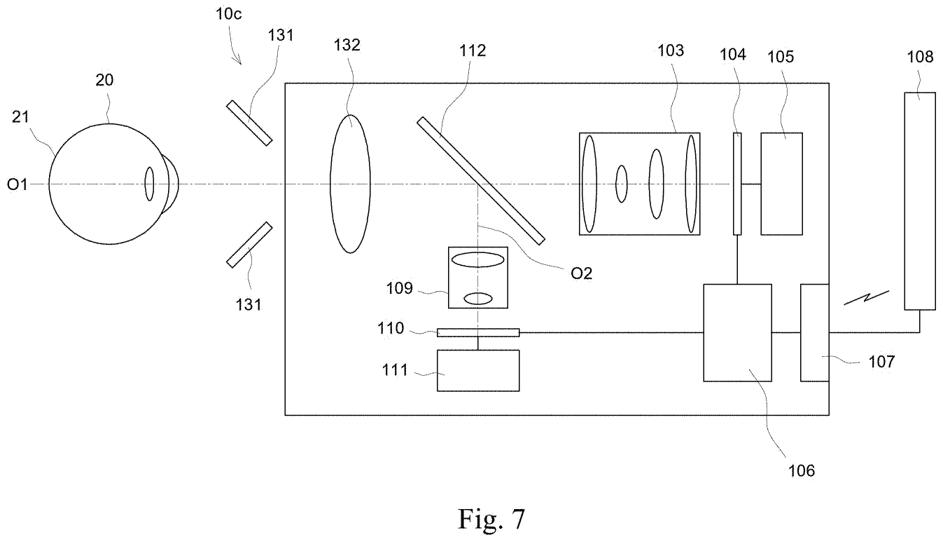

Refer to FIG. 7 for a fundamental structure of a corneal topography device 10c. The main functional elements of the corneal topography device 10c include a patterning illumination element 131 and an object lens 132. The patterning illumination element 131 is used to generate a patterned illumination light beam having a specified pattern and project the patterned illumination light beam to a tested eyeball 20. The surface of the tested eyeball 20 reflects the light beam. The reflected light beam passes through the object lens 132 and the imaging lens assembly 103 to the image sensor 104 and form an image of the eyeball surface. The defects of the eyeball surface may be identified according to the deformation of the pattern. The other elements of the corneal topography device 10c shown in FIG. 7, which have the same numeral symbols as the elements in FIG. 1, also have the same functions as these elements in FIG. 1. Since the technical contents thereof has been described before, it will not repeat herein. The persons having ordinary knowledge of the art should be able to modify the pupil tracking method and the ophthalmology inspection devices without departing from the spirit of the present invention.

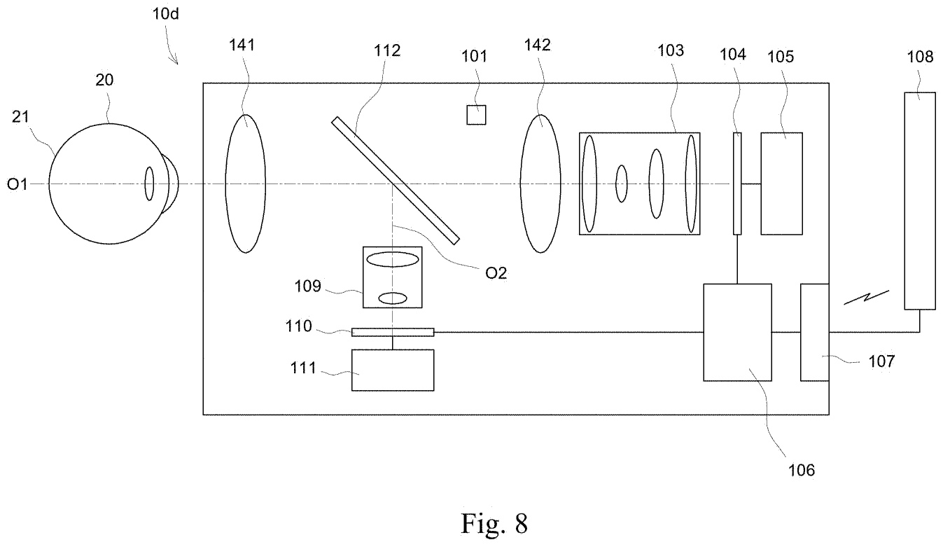

Refer to FIG. 8 for a fundamental structure of an automatic refractometer 10d. The main functional elements of the automatic refractometer 10d include an object lens 141 and a feature optical element 142. The feature optical element 142 generates an annular feature-type light source. While the annular light source is projected onto the fundus, the automatic refractometer 10d can estimate the diopter of the tested eyeball 20 according to the shape of the annular light source. The internal display device 110 may present an optometric pattern. Adjusting the focal length between the internal display device 110 and the display lens assembly 109 may induce the eyeball of the subject to have accommodation relax. For example, the focal length may be changed from 10 cm equivalently to infinity equivalently via using the display focal length-adjusting element 111 to adjust the internal display device 110. A fogging lens may be used to make the subject view a blurred optometric image and induce the eyeball of the subject to relax. Then, the diopter of the tested eyeball 20 is inspected. The other elements of the automatic refractometer 10d shown in FIG. 8, which have the same numeral symbols as the elements in FIG. 1, also have the same functions as these elements in FIG. 1. Since the technical contents thereof has been described before, it will not repeat herein. The persons having ordinary knowledge of the art should be able to modify the pupil tracking method and the ophthalmology inspection devices without departing from the spirit of the present invention.

In conclusion, the ophthalmology inspection device and the pupil tracking method of the present invention may find out a pupil feature from the external eye image and may use a boundary fitting method to find out a center coordinate of the pupil feature. Therefore, the ophthalmology inspection device can track the pupil of the eyeball of a subject without using a stereo camera. In a preferred embodiment, the ophthalmology inspection device of the present invention can work out the deviation between the imaging optical axis of the ophthalmology inspection device and the pupil of the subject according to the center coordinate of the pupil feature. Then, the ophthalmology inspection device generates an indication signal to assist the operator or the subject in adjusting the relative position of the ophthalmology inspection device and the pupil of the subject. Thereby, the ophthalmology inspection device is exactly aligned to the pupil of the subject to acquire a better inspection result.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. A pupil tracking method comprising steps:
using an ophthalmology inspection device to acquire an external eye image of a subject, wherein the external eye image includes a pupil of the subject;
using the ophthalmology inspection device to perform an image preprocessing on the external eye image, wherein the image preprocessing includes performing a binary conversion on the external eye image to obtain a binary image;
finding out a contour boundary of each feature in the binary image, and finding out a pupil feature based on a variance of a distance from the contour boundary of each feature to a corresponding reference point; and
fitting the contour boundary of the pupil feature in a boundary fitting method to find a center coordinate of the pupil feature.

2. The pupil tracking method according to claim 1 further comprising a step:
the ophthalmology inspection device generating an indication signal to remind the subject or an operator to adjust a relative position of the ophthalmology inspection device and the subject to make the ophthalmology inspection device be exactly aligned to the pupil of the subject.

3. The pupil tracking method according to claim 1, wherein the image preprocessing further includes a step: reducing a size of the external eye image before the binary conversion to reduce a computation amount of succeeding image processing steps.

4. The pupil tracking method according to claim 1, wherein the image preprocessing further includes a step: performing a noise reduction treatment of the external eye image before the binary conversion to eliminate grain-like noises of the external eye image.

5. The pupil tracking method according to claim 4, wherein the noise reduction treatment is realized by an average filter, a Gaussian filter, a median filter, or a bilateral filter.

6. The pupil tracking method according to claim 1, wherein the image preprocessing further includes a step: performing an image enhancing treatment of the external eye image before the binary conversion to enhance image boundaries.

7. The pupil tracking method according to claim 6, wherein the image enhancing treatment is realized by a gamma correction technology, a histogram equalization technology, a homomorphic filter technology, an unsharp masking technology, or a combination thereof.

8. The pupil tracking method according to claim 1, wherein the image preprocessing further includes a step: performing an opening operation of morphology on the binary image to compensate for defects or small-area noises generated in the binary conversion.

9. The pupil tracking method according to claim 1, wherein the reference point is a center of a minimum enclosing circle fitting the contour boundary of each feature.

10. The pupil tracking method according to claim 1, wherein while the variance of a feature is smaller than or equal to a preset value, the feature is the pupil feature.

11. The pupil tracking method according to claim 1, wherein the boundary fitting method may be a circle fitting method, an ellipse fitting method, or a minimum enclosing circle method.

12. The pupil tracking method according to claim 1, wherein a least square method is used to find out the center coordinate of the pupil feature.

13. An ophthalmology inspection device comprising
an illumination element, generating an illumination light beam to illuminate an external eye region of a subject;
an image sensor, receiving a light beam light reflected from the external eye region to generate an external eye image, wherein the external eye image includes a pupil of the subject; and
an imaging lens assembly, disposed at a light-input side of the image sensor to condense the light beam reflected from the external eye region and form an image to the image sensor; and
a signal processing element, electrically connected with the image sensor, wherein the signal processing element performs a pupil tracking method, which comprises steps:
acquiring the external eye image output by the image sensor;
performing an image preprocessing on the external eye image, wherein the image preprocessing includes performing a binary conversion on the external eye image to obtain a binary image;
finding out a contour boundary of each feature in the binary image, and finding out a pupil feature based on a variance of a distance from the contour boundary of each feature to a corresponding reference point; and
fitting the contour boundary of the pupil feature in a boundary fitting method to find a center coordinate of the pupil feature, and calculating a deviation between an optical axis of the imaging lens assembly and the pupil of the subject according to the center coordinate of the pupil feature.

14. The ophthalmology inspection device according to claim 13 further comprising
an internal display device, electrically connected with the signal processing element, wherein the signal processing element presents an indication signal on the internal display device according to the deviation;
a display lens assembly, disposed at a light-output side of the internal display device;
a display focal length-adjusting element, connected with the internal display device or the display lens assembly to adjust a focal length of the indication signal;
a light splitter, optically coupled to the internal display device and the imaging lens assembly, imaging the indication signal on a fundus of the subject to remind the subject to adjust a relative position of the ophthalmology inspection device and the subject to make the ophthalmology inspection device be exactly aligned to the pupil of the subject.

15. The ophthalmology inspection device according to claim 13 further comprising
an external display device, coupled to the signal processing element, wherein according to the deviation, the signal processing element presents an indication signal on the external display device to remind an operator to adjust a relative position of the ophthalmology inspection device and the subject to make the ophthalmology inspection device be exactly aligned to the pupil of the subject.

16. The ophthalmology inspection device according to claim 13 further comprising
an imaging focal length-adjusting element, connected with at least one of the image sensor and the imaging lens assembly to physically move the image sensor or at least one lens of the imaging lens assembly or adjust a curvature of a liquid-state lens of the imaging lens assembly to make the light beam reflected from the external eye region be imaged on the image sensor.

17. The ophthalmology inspection device according to claim 13, which is a fundus camera, a tonometer, a corneal topography device, or an automatic refractometer.

18. The ophthalmology inspection device according to claim 13, wherein the image preprocessing further includes a step: reducing a size of the external eye image before the binary conversion to reduce a computation amount of succeeding image processing steps.

19. The ophthalmology inspection device according to claim 13, wherein the image preprocessing further includes a step: performing a noise reduction treatment of the external eye image before the binary conversion to eliminate grain-like noises of the external eye image.

20. The ophthalmology inspection device according to claim 19, wherein the noise reduction treatment is realized by an average filter, a Gaussian filter, a median filter, or a bilateral filter.

21. The ophthalmology inspection device according to claim 13, wherein the image preprocessing further includes a step: performing an image enhancing treatment of the external eye image before the binary conversion to enhance image boundaries.

22. The ophthalmology inspection device according to claim 21, wherein the image enhancing treatment is realized by a gamma correction technology, a histogram equalization technology, a homomorphic filter technology, an unsharp masking technology, or a combination thereof.

23. The ophthalmology inspection device according to claim 13, wherein the image preprocessing further includes a step: performing an opening operation of morphology on the binary image to compensate for defects or small-area noises generated in the binary conversion.

24. The ophthalmology inspection device according to claim 13, wherein the reference point is a center of a minimum enclosing circle fitting the contour boundary of each feature.

25. The ophthalmology inspection device according to claim 13, wherein while the variance of a feature is smaller than or equal to a preset value, the feature is the pupil feature.

26. The ophthalmology inspection device according to claim 13, wherein the boundary fitting method may be a circle fitting method, an ellipse fitting method, or a minimum enclosing circle method.

27. The ophthalmology inspection device according to claim 13, wherein a least square method is used to find out the center coordinate of the pupil feature.
